(19) United States
(12) Patent Application Publication
(10) Pub. No.: US 2025/0268667 A1
(43) Pub. Date: Aug. 28, 2025

(54) MEDICAL KINEMATIC SYSTEM HAVING A VIRTUAL FULCRUM, MEDICAL ROBOT, AND USE OF A MEDICAL KINEMATIC SYSTEM AND A MEDICAL ROBOT

(71) Applicant: KARL STORZ SE & Co. KG, Tuttlingen (DE)

(72) Inventor: Martin HOFFMANN, Tuttlingen (DE)

(73) Assignee: KARL STORZ SE & Co. KG, Tuttlingen (DE)

(21) Appl. No.: 18/848,313

(22) PCT Filed: Mar. 21, 2023
(86) PCT No.: PCT/EP2023/057180

§ 371 Date: May 16, 2025

(30) Foreign Application Priority Data
Mar. 21, 2022 (DE) 10 2022 106 602.9

(51) Int. Cl. A61B 34/30

(52) U.S. Cl. CPC A61B 34/30; A61B 2034/301

(57) ABSTRACT

The invention relates to a medical kinematic system having a virtual fulcrum (RCM) for a medical robot for guiding a surgical instrument arranged on an instrument holder within a working zone, having a main body, a first linear drive, and a second linear drive for movably arranging an instantaneous fulcrum of the instrument holder within the working zone and simultaneously pivoting the instrument holder about the instantaneous fulcrum. The invention also relates to a medical robot having such a kinematic system and to a method for operating a medical kinematic system or a medical robot.

### CROSS-REFERENCE TO RELATED APPLICATIONS

This application is the U.S. national stage of PCT/EP2023/057180 filed on Mar. 21, 2023, which claims priority of German Patent Application No. DE 10 2022 106 602.9 filed on Mar. 21, 2022, the contents of which are incorporated herein.

### TECHNICAL FIELD

The invention relates to a medical kinematic system having a virtual fulcrum (RCM) for a medical robot for guiding a surgical instrument arranged on an instrument holder within a working zone, having a main body, a first linear drive, and a second linear drive for movably arranging an instantaneous fulcrum of the instrument holder within the working zone and simultaneously pivoting the instrument holder about instantaneous fulcrum. Furthermore, the invention relates to a medical robot having such a kinematic system and to a method for operating a medical kinematic system or a medical robot.

### BACKGROUND

Known medical kinematic systems, such as those known from DE 10 2018 118 066 A1, use different linear drives to generate a virtual fulcrum. Different linkage mechanisms are guided in a curved shape, e.g., with a plurality of guides, in order to become effective on an instrument holder via an interaction of the movements of these mechanisms and the curved shapes. The aim of these arrangements is to rotate a medical instrument about a virtual fulcrum. This process often has to take place in a small space, but a clearly defined virtual fulcrum must still be realized—for example, at an entry point on a body. Further robotic kinematic systems for generating a virtual fulcrum are known from US 2015/0351857 A1 and DE 102013004459 A1.

### SUMMARY

The object of the invention is to improve upon the prior art.

The object is achieved by a medical kinematic system having a virtual fulcrum (RCM) for a medical robot for guiding a surgical instrument arranged on an instrument holder in a working zone, with a main body, a first linear drive, and a second linear drive for the movable arrangement of an instantaneous fulcrum of the instrument holder in the working zone and simultaneous pivoting of the instrument holder about the instantaneous fulcrum, wherein the first linear drive has a fixed point, for receiving a first linear drive part of the first linear drive on the main body, and a first guide point for guiding a second linear drive part of the first linear drive relative to the main body in a first guide track, and the second linear drive has a second fixed point, for receiving a first linear drive part of the second linear drive, and a second guide point for guiding the second linear drive part of the second linear drive relative to the main body and/or relative to the second linear drive part of the first linear drive in a second guide track, wherein one of the guide tracks has a guide cam, and at least the linear drive guided in the guide track other than the guide track with the guide cam has a has a variable transmission ratio, so that, by means of a transmission arrangement acting mechanically between the first linear drive and the second linear drive and by means of an interaction of a geometry of the guide cam with the at least one variable transmission ratio, the instantaneous fulcrum can be moved in a circular path about the virtual fulcrum, and the instrument holder can be pivoted about the virtual fulcrum.

Such a medical kinematic system can be built very compactly due to the linear drives being arranged spatially close to one another and assigned to one another, wherein it is ensured in particular by means of the movable arrangement of an instantaneous fulcrum that the virtual fulcrum is held particularly securely and precisely solely as a result of the geometric relationships of the mechanical components—in this connection, the design of the guide cam in connection with the transmission ratio the transmission of arrangement. In particular, a further guide cam for the corresponding other linear drive can be omitted and replaced by the variable transmission ratio.

In this connection, the following terms are explained:

A “medical kinematic system” describes a combination of components that are connected to one another in a movable or partially also immovable manner, which serves, for example, to guide a medical instrument and to position it spatially, wherein, here, positioning can include both a displacement along linear axes in space and a rotation about corresponding axes. Such a medical kinematic system is realized, for example, as an articulated rod structure or the like, but can also have ball joints, pivot joints, or the like.

Such a medical kinematic system has in particular a “virtual fulcrum,” which is also referred to as an “RCM,” i.e., “remote center of motion.” This means in particular that such a virtual fulcrum is not, or is only in certain cases, arranged directly in the region of a mechanical fulcrum, such as a joint, but that the medical kinematic system works in such a way that a spatially fixed point, viz., the virtual fulcrum, remains stationary although the medical kinematic system is executing arbitrary movements. Such a virtual fulcrum is used in particular to form a so-called “trocar point,” viz., an entry point—for example, into a human body during a minimally invasive operation. For example, a trocar, i.e., a sheath of a medical instrument, can be guided in such a way that lateral forces on the entry region are avoided or prevented. Nevertheless, it is possible to move a medical instrument within the trocar or another medical or surgical instrument—for example, by rotational degrees of freedom. It is also possible to insert or withdraw an instrument, for example, within the trocar through the virtual fulcrum without exerting lateral forces on, for example, the skin of the patient.

A “medical robot” is usually a multi-axis robot that is used for telesurgical interventions, i.e., partially or completely machine-assisted surgical procedures. Such a medical robot can be designed as desired as an articulated arm robot, a linear axis robot, or a robot of some other design. It is also pointed out that the mechanical boundaries between the medical robots as a movable fixed point for a medical kinematic system and the medical kinematic system itself can be fluid; in particular, both components of a medical system can merge into one another or be part of one another. In conceptual terms, the medical robot serves in particular as a movable platform for the global movement of a medical instrument, and the medical kinematic system is often used for fine adjustment, e.g., for a fine positioning, also with the aforementioned virtual fulcrum.

In this connection, “guiding” is carried out in such a way that the surgical or medical instrument can be positioned in a defined manner; for example, it can be moved along axes or rotated about axes. Such guidance can be carried out entirely or partially manually, but can also be electronically and/or technically supported—for example, by means of a remote control, a joystick, or other control elements.

An “instrument holder” describes the part of the medical kinematic system in which a surgical instrument is held—for example, as an exchangeable surgical instrument with a corresponding interface. Accordingly, an instrument holder is, for example, a mechanical component which is arranged as the last component on the medical kinematic system in the direction of the body to be treated, before the surgical instrument can be connected in the distal direction.

A “surgical instrument” here can, for example, be a laparoscope, an endoscope, or a surgical instrument which is to be guided by means of the medical kinematic system in relation to, for example, a patient. In particular, such a surgical instrument can also have or provide means for preparing and/or following up on an operation, such as adhesive, stapling, or suturing aids, or even cutting tools.

In this connection, a “working zone” describes the region in which, for example, an operator works using the medical kinematic system and the surgical instrument. For example, the working zone is a region of a work table or a treatment table, a patient lying on it, and a correspondingly suitable environment for preparing and, for example, pre-positioning the medical kinematic system and/or surgical instrument. In particular, such a working zone is not strictly defined, but, rather, arises from the context—for example, an operation to be performed.

The medical kinematic system has a “main body” on which the remaining components of medical kinematic system are arranged. Such a main body is in particular also provided with a coupling point in the direction of a medical robot or can also be a last component of such a medical robot at a distal end of the medical robot, starting from a medical robot in the direction of the medical kinematic system; here, the boundaries can be fluid.

A “linear drive” associated with a medical kinematic system is a technical device that can initiate a movement along a particular linear axis. Such a linear drive is, for example, a linear motor, a linear guide, a linearly guided spindle drive, or the like. The term linearity is to be understood in such a way that technically reasonable deviations are to be tolerated, so that for example a linear drive can also be arranged and equipped in a slightly curved or wave-shaped manner or in a similar way. In this connection, a mathematically precise linearity of the drive movement is not necessary and/or, depending upon the spatial requirements, may even expressly not be required. A linear drive has a first “linear drive part” and a second “linear drive part,” wherein the first linear drive part is, for example, stationary, and the second linear drive part is, for example, movable relative to the first linear drive part.

By means of one of the linear drives, a so-called “instantaneous fulcrum” is arranged movably in the working zone, where such an instantaneous fulcrum is designed analogously to the virtual fulcrum, where the “instantaneous fulcrum” exists within the medical kinematic system and is therefore fixedly arranged in relation to other components, e.g., to components of the first linear drive; in contrast to the virtual fulcrum, however, this instantaneous fulcrum does not have to be fixed in relation to the working zone, but is arranged movably in it. This instantaneous fulcrum is, for example, a joint, a bearing, or an analogous arrangement about which further components can then be pivoted.

In this connection, a “pivoting” of the instrument holder describes a rotational movement of the instrument holder about the instantaneous fulcrum, wherein a translational movement of the instrument holder can also be the result of a movement of the instantaneous fulcrum. The relation is established, for example, between the instrument holder and the instantaneous fulcrum in such a way that the instantaneous fulcrum serves as the rotation point of the instrument holder. Such a pivoting occurs in particular in one plane.

The linear drive has a “fixed point,” wherein this fixed point exists in relation to the main body or in relation to the other linear drive and enables a rotationally fixed or, in particular, a rotationally movable holding of a first linear drive part. For example, this first “linear drive part” is a fixed component of the linear drive, wherein it is pivotably accommodated on the fixed point, and a further linear drive part represents the movable part of the linear drive. If such a linear drive is designed as a spindle drive, the spindle of the linear drive would, for example, be fixed in the axial direction, but pivotable with a spindle axis in a plane or held on the fixed point so as to be rotatable about it, and a corresponding component with a spindle nut would represent the movable linear drive part of the linear drive.

In this case, a “guide point,” which is assigned to the further linear drive part—in the example shown, the movable linear drive part with spindle nut—is guided along a “guide track.” This guide track serves to spatially guide the guide point when the linear drive part is extended or shortened. If a fixed point is designed to be rotatable so that the linear drive can rotate about the fixed point during its operation from a shortened to an extended position or in a reverse movement, the linear drive can perform a guided, e.g., oscillating, wave-like movement. The guide track then has a “guide cam” with a non-rectilinear “geometry,” which describes, for example, the curved or arcuate course of the guide cam. In this connection, it should be mentioned that in particular a mechanical coupling of the guide track and/or the guide cam to the guide point is carried out in such a way that a forced guidance takes place.

A “circular path,” which results as the line of movement of the instantaneous fulcrum about the virtual fulcrum, is in this connection not to be understood exclusively as a mathematically exact circular path, i.e., as a circular curve in the mathematical sense, but serves as an example for illustration and can have correspondingly tolerable deviations. In this connection, the circular path is defined in such a way that the virtual fulcrum is maintained as a fixed point in space within the correspondingly necessary tolerances. Likewise, some other mathematical function can be represented as a circular path or as an alternative to a circular path, e.g., an elliptical path, a parabolic path, or any other, particularly closed, path or trajectory, in each case with physically determined deviations from the mathematical ideal state.

Between the first linear drive and the second linear drive, a so-called “transmission arrangement” acts which has a variable “transmission ratio” and couples the first linear drive and the second linear drive in such a way that a mechanical interaction, i.e., a simultaneous operation of the second linear drive, takes place when, for example, the first linear drive is driven from the outside. The “variable transmission ratio” here describes a property of an individual linear drive such that, for example, along a length of movement of the linear drive, the linear drive is driven with a transmission ratio that can be modified, i.e., when there is a uniform drive of the linear drive, its linear movement is carried out according to a non-constant or even non-linear function. A transmission arrangement can be realized, for example, with a positive drive in the form of a toothed belt, in the form of gears, or a gear mechanism acting in some other way between the linear drives, wherein these or other parts of the transmission arrangement can then also realize the variable part of the transmission. For example, a toothed belt can be used to drive a threaded spindle with a fixed transmission ratio, wherein the threaded spindle itself then has a variable pitch in order to achieve the variable transmission ratio. Alternatively, an electronic coupling can also be realized via, for example, a corresponding synchronization of stepper motors with a variable transmission ratio stored in a controller, provided that this is expedient, for example, with regard to the installation space or the arrangement of the components.

In this connection, it should be mentioned that the variable transmission ratio is selected on the basis of the spatial and geometric conditions in such a way that, due to the interaction of the geometry of the guide cam with the variable transmission ratio, the instrument holder can be pivoted about the virtual fulcrum at any point of a corresponding pivoting movement, and the virtual fulcrum is thus arranged immovably in space within the desired tolerances.

In one embodiment, the first guide track assigned to the second linear drive part of the first linear drive can have the guide cam, wherein in particular the second guide track assigned to the second linear drive part of the second linear drive is designed to be substantially rectilinear. In a further embodiment, the second guide track assigned to the second linear drive part of the second linear drive has the guide cam, wherein in particular the first guide track assigned to the second linear drive part of the first linear drive is substantially linear, and in particular the instrument holder is received on the second linear drive part so as to be movable relative to the second linear drive part of the first linear drive substantially in one direction through the virtual fulcrum.

Thus, in different embodiments and, for example, depending upon the available installation space, either the first linear drive or the second linear drive can be equipped with the guide cam, wherein the other linear drive is guided in a straight line.

In order to achieve a smooth transition of the movements and to be able to construct the medical kinematic system as simply as possible, the geometry of the guide cam is arcuate, parabolic, and/or elliptical, wherein a center point of the curvature of the geometry and/or a center point of the curvature of the relevant portion of the geometry, i.e., the arcuate portion, the parabolic portion, and/or the elliptical portion, is arranged starting from the geometry in the direction of the virtual fulcrum.

In this connection, it has been found that such an arrangement can be implemented in a particularly compact manner, which means that, for example, the naturally very limited space in the workspace near a patient during an operation can be used very efficiently.

The terms “arcuate,” “parabolic,” and/or “elliptical” describe a design in accordance with the respective mathematical designation, but expressly cover corresponding technically required tolerances. In this connection, the guide cam can also be composed of different arcuate, parabolic, and/or elliptical components, so that, overall, a continuous and differentiable guide cam is created for a uniform and jerk-free guidance of the surgical instrument.

A “center of curvature” describes the current center of the circular path of a correspondingly curved section of the guide cam at each point of the curve.

In one embodiment, the guide cam has at least one sliding link, wherein the guide point has a pin guided in the at least one sliding link. As an alternative to a pin engaging in a sliding link, a guide clamp engaging over a guide rail can also be used if this is indicated, for example, for reasons of simpler production.

The advantage f such a sliding link with a corresponding pin or an analogous design is the forced guidance of the components along the guide cam, so that, in a compact mechanical arrangement, a deviation of the movement of the medical kinematic system from a movement about the virtual fulcrum is almost impossible.

In this connection, a “sliding link” describes a gear element which usually has a slot, a web, or a groove and guides a pin guided in or on the link, which is also called a slide block, or an alternative guide means, and thus impresses a predetermined movement path along the longitudinal extent of the link.

For example, such a sliding link is designed as a slot formed in a flat component along its longitudinal direction, wherein a corresponding “pin” can, for example, be a round pin, which is then ideally guided without play within the sliding link.

In order to be able to implement the medical kinematic system in a particularly compact manner, the second linear drive is arranged to act substantially parallel to the first linear drive, and/or a first linear drive part of the second linear drive is assigned to the second linear drive part of the first linear drive, wherein in particular the second linear drive part of the second linear drive is driven via the transmission arrangement with the variable transmission ratio.

“Substantially parallel” describes a fundamentally parallel action, wherein physically caused deviations of, for example, ±10° or ±15° are also covered. In this connection, angle specifications refer to a full angle of 360 degrees.

In one embodiment, a lever is assigned to the second linear drive part of one of the linear drives, wherein the lever is connected to the instrument holder in a rotatable, mechanically acting manner when the instrument holder is pivoted about the instantaneous fulcrum by means of the corresponding linear drive.

Such a lever can be designed with, for example, a first lever fulcrum and a second lever fulcrum acting in the sense of a coupling rod, such that the corresponding arrangement is part of the transmission ratio, so that a particularly compact design of the medical kinematic system is enabled.

A “lever” in this connection can for example be a web with a first fulcrum and a second fulcrum, which acts in a function analogous to a push rod or a coupling rod with a holder that can be moved on both sides.

In order to enable corresponding assemblies, e.g., for producing different medical kinematic systems with different effective sizes, the variable transmission ratio is realized by means of a drive ratio of the first linear drive and/or the second linear drive that can be changed over a length of movement of the second linear drive.

For example, the second linear drive can be designed with a drive ratio that can be changed over its length of movement, so that a corresponding combination of linear drives with corresponding standard assemblies enables a different arrangement in each case of a virtual fulcrum in relation to the main body of the medical kinematic system. This greatly simplifies the production of different medical kinematic systems, e.g., for different movement requirements.

In one embodiment, one of the linear drive parts of the corresponding linear drive has a spindle with pitch flanks arranged in a beam shape along the length of movement and a pitch of the pitch flanks that is variable over the length of movement, wherein the other linear drive part of the corresponding linear drive is assigned a spindle nut guided on the spindle with engagement elements engaging in the pitch flanks.

A “spindle” is, for example, a thread-like, rod-shaped arrangement which has “pitch flanks” made in this spindle, which are made analogously to thread turns. Corresponding “engagement elements” engage in this “pitch flank,” which are, for example, in the form of sliding pieces designed to correspond to a cross-sectional geometry of the pitch flanks.

In order to be able to use such a spindle with a corresponding spindle nut with as little play as possible, which is particularly important in medical kinematic systems, the spindle nut has elastic compensating means acting between the engagement elements to compensate for distance differences occurring along the length of movement, the variable geometry in each case due to the variable pitch, and/or tolerances between and/or on the respective pitch flanks.

Such an “elastic compensating means” can for example be a spring or a spring element, wherein different engagement elements are braced against each other by means of the elastic compensating means and act in each case in an effective direction of the spindle, and can thus compensate for distance differences and/or tolerances. Mechanical springs as well as hydraulic spring devices or equivalent means can be used.

In one embodiment, a third linear drive with an axis of movement running through the virtual fulcrum is assigned to the instrument holder for the linear movement of the surgical instrument through the virtual fulcrum.

Such a third linear drive can, for example, achieve a depth of penetration of the surgical instrument into a body to be treated in such a way that the virtual fulcrum remains unchanged, which is why the third linear drive has an axis of movement that substantially runs through the virtual fulcrum. In this connection, it should also be noted that physically caused deviations are expressly included.

Likewise, the main body can be assigned a rotary holder with an axis of rotation for the rotationally movable connection of the medical kinematic system to a medical robot, wherein the axis of rotation is arranged skewed to an axis of movement of the first linear drive.

By means of such a rotary holder with a corresponding axis of rotation, a substantially two-dimensional mode of action due to the previous design of the medical kinematic system can be extended to a third dimension, wherein the arrangement of the axis of rotation is in particular such that the axis of rotation runs through the virtual fulcrum. This allows movement of the medical kinematic system in all six degrees of freedom, viz., three translational and three rotational degrees of freedom.

In a further aspect, the object is achieved by a medical robot having a medical kinematic system according to one of the previously described embodiments. Such a medical robot utilizes all the advantages of the medical kinematic system and can also position and guide the medical kinematic system globally in the workspace or outside the workspace, providing an overall easy-to-use, compact medical system.

In a further aspect, the object is achieved by a method for operating a medical kinematic system according to one of the previously designated embodiments and/or a medical robot with a virtual fulcrum, having the following steps: arranging the medical kinematic system in the working zone so that the medical kinematic system is arranged in the working zone, guiding the medical kinematic system and/or the medical robot about the virtual fulcrum (RCM) by means of an interaction of the geometry of the guide cam with the variable transmission ratio, so that the instrument holder is pivoted about the virtual fulcrum, so that the medical kinematic system is operated with a virtual fulcrum.

The invention is explained below with reference to exemplary embodiments.

### BRIEF DESCRIPTION OF THE DRAWINGS

FIG. 1 is a schematic representation of a kinematic system for a medical robot in a side view,

FIG. 2 is a schematic overview of a medical robot with a kinematic system of FIG. 1,

FIGS. 3a to 3c show the kinematic system of FIG. 1 in different positions, and

FIG. 4 is a schematic detailed representation of a spindle drive of the kinematic system of FIG. 1.

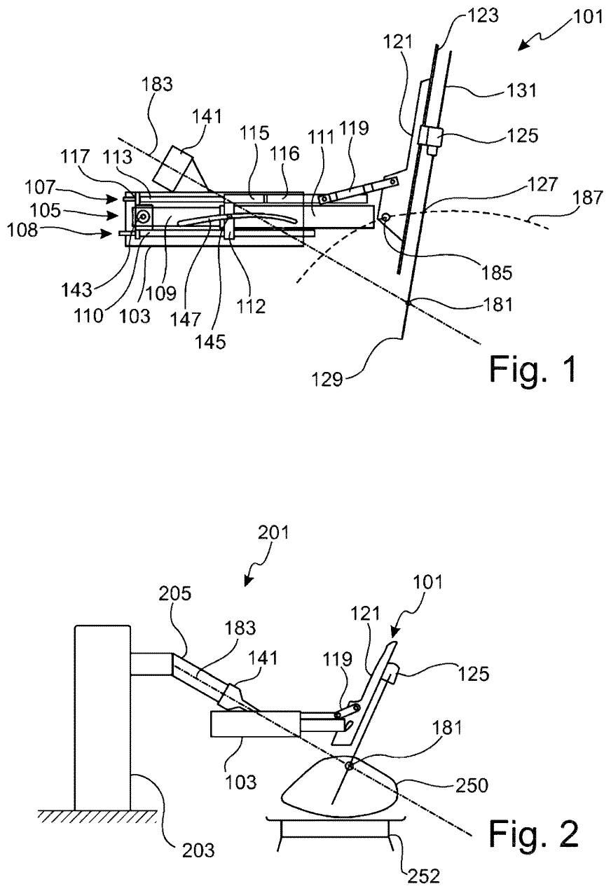

### DETAILED DESCRIPTION OF PREFERRED EMBODIMENTS

A kinematic system 101 for medical purposes is a so-called “RCM” mechanism, which can be used to guide a medical instrument. The kinematic system 101 has a main body 103. The main body 103 serves to hold the other components of the kinematic system 101 and thus also acts as a mechanical reference system for the movement of other components. The main body 103 can be connected to a medical robot by means of a rotary joint 141, wherein rotation about an axis of rotation 183 of the rotary joint 141 in relation to the medical robot is possible.

A guide 105 is arranged on the main body 103, wherein a static part 109 of the guide is connected rotatably but in stationary fashion to the main body 103 by means of a fixed point 143. A movable part 111 of the guide 105, which is arranged to be longitudinally displaceable relative to the static part 109, can slide back and forth on the static part 109 in relation to the fixed point 143. A guide point 145, which is designed as a pin, is arranged on the movable part 111. This guide point 145 slides in a link 147, wherein the link 147 is arranged on the main body 103. The link 147 is arcuate in shape, wherein a longitudinal displacement of the movable part 111 relative to the static part 109 of the guide 105 initiates a corresponding oscillating movement of the guide 105.

A linear drive 108 is used to drive the guide 105, wherein a spindle 110 is fixedly arranged axially relative to the fixed point 143, and a movable part 112 is, mechanically, fixedly connected axially to the movable part 111 of the guide 105. Thus, the movable part 111 of the guide 105 can be moved axially by means of the linear drive 108, thereby triggering the oscillating movement described above. The guide 105 serves only to absorb mechanical forces and is thus part of the linear drive 108 in the sense of its functional purpose, viz., the drive in a linear direction.

Also associated with the guide 105 is a linear drive 107, wherein a spindle 113 of the linear drive 107 is also rotatable via a bearing point 117, but is fixedly arranged axially relative to the fixed point 143. A movable part 116 which is driven by the spindle 113 is axially displaceable, analogously to the previously described processes, parallel to the guide 105 and linear drive 108. For this purpose, the movable part 116 is accommodated in a support tube 115 so as to be longitudinally movable, in particular in order to avoid tilting relative to the spindle 113 and in order to guide the movable part 116.

The linear drive 107 here has a variable transmission ratio, to be explained in detail in later embodiments.

Between the linear drive 108 and the linear drive 107, there is a mechanical coupling in the form of a toothed belt transmission (not shown), so that a rotary movement of the linear drive 108 is synchronized with a rotary movement of the linear drive 107. Overall, this mechanical coupling and the variable transmission of the linear drive 107 over its length of movement result in a variable transmission ratio between the linear drive 108 and the linear drive 107.

The guide 105 serves to move an instantaneous fulcrum 185 in space. An instrument holder 121 is arranged so as to pivot about the instantaneous fulcrum 185. The instrument holder 121 has a running rail 123 on which a linear drive 125 is arranged. By means of the linear drive 125, a shaft 127 as part of a surgical instrument is accommodated on the guide rail 123 in a longitudinally displaceable manner. The shaft 127 also has supply line 131, which here stands as an example for instruments, lighting devices, and the like guided in the trocar 127. The shaft 127 also has an instrument tip 129.

The linear drive 107, in particular the movable part 116 of the linear drive 107, is provided with a lever 119. The movable part 116 is guided in the support tube 115 and is connected to the instrument holder 121 by means of the lever 119 in such a way that the instrument holder 121 is driven by means of the linear drive 107 via the lever 119. The linear movement of the linear drive 107 acts upon the instrument holder 121 by means of the lever 119 and the further transmission participation resulting from the arrangement of the lever. Overall, the linear drive 107 and the lever 119 thus form a pivot drive for the instrument holder 121. Thus, the instrument holder 121 can be pivoted about the instantaneous fulcrum 185 by means of the linear drive 107 and the coupling action of the lever 119.

The geometric relationships are explained again in detail below:

For, for example, surgical procedures, it is useful for a surgical instrument to rotate about a virtual fulcrum, i.e., to not execute any lateral movement at, for example, an entry point into a body. In this connection, the shaft 127 has a so-called trocar point, viz., the virtual fulcrum 181, at which the shaft 127 is inserted, for example, in a trocar. The link 147, which acts upon the pin 145, is realized in connection with the transmission of the linear drive 108 to the linear drive 107 and the variable transmission ratio over the length of movement of the linear drive 107 in cooperation with the lever 119 in such a way that the instantaneous fulcrum 185 describes a circular path about the virtual fulcrum 181 when the linear drive 108 is driven, viz., the circular path 187. Likewise, the angle of the axis of rotation 183 to the corresponding movement axes of the linear drives is set to be skewed in such a way that the axis of rotation 183 passes through the virtual fulcrum 181. Thus, a movement of the instrument using the kinematic system 101 and a rotation of the kinematic system 101 about the axis of rotation 183, e.g., on a medical robot, can be carried out in such a way that the virtual fulcrum 181 remains fixed in space—for example, also fixed in relation to an entry point on a patient.

By way of example, a medical robot 201 with a foot 203 and an arm 205 is described, wherein the kinematic system 101 is connected to a distal end of the arm 205. The virtual fulcrum 181 is fixedly located at a boundary of an exemplary body 250, which is lying on a table 252—for example, an operating table.

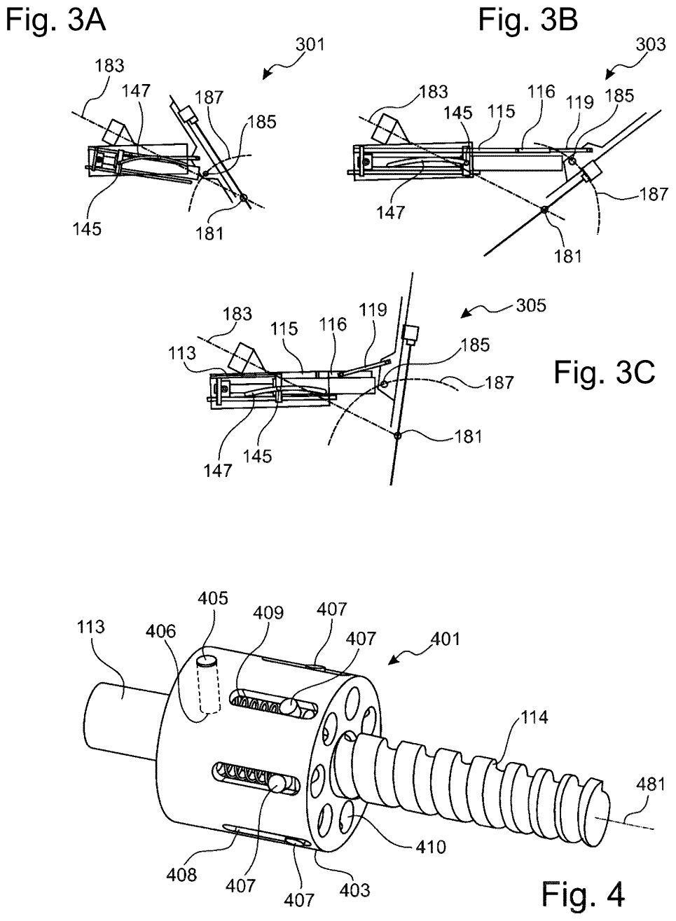

The action of the kinematic system 101 is shown again in detail (cf. also FIGS. 3a to 3c):

Regardless of the position of the linear drive 108 and the linear drive 107 in relation to the main body 103 of the kinematic system 101, a movement of the instantaneous fulcrum 185 on the circular path 187 about the virtual fulcrum 181 can be observed in different positions 301, 303, and 305 (cf. FIGS. 3a, 3b, 3c). The pivoting movement of the instrument holder 121 is tracked by means of the linear drive 107 in such a way that no physically harmful deviations from this movement occur. A rotation of the kinematic system 101 about the axis of rotation 183 also has no negative influence here.

A spindle drive 401, as used in the linear drive 107, is explained here again in detail:

The spindle drive 401 has the spindle 113 with spiral, thread-like flanks 114, wherein the corresponding flanks are part of a U-shaped profile.

Corresponding to the profile of these flanks 114, a pin 405 is guided radially to the spindle 113 within a spindle nut 403 and engages in a flank 114 with a head region 406. This creates a fixed longitudinal relation along a longitudinal axis 481 through the pin 405. The pin 405 serves as a pin fixedly arranged relative to the spindle nut 403. Further pins 407, which are arranged in the spindle nut 403 in a rotationally fixed but longitudinally displaceable manner in elongated holes and are also arranged radially to the spindle 113 and positioned so as to engage in the flanks 114, are held free of play in relation to the pin 405 by means of springs 409, so that the pins 405 and 407 are braced within the flanks 114 by means of the springs 409, thus enabling the spindle nut 403 to be guided free of play on the spindle 113.

The variable pitch of the flanks 114 along a longitudinal axis 481 of the spindle 113 (cf. FIG. 4) is part of the variable transmission ratio of the linear drive 107 compared to the linear drive 108. The pins 405, together with the pins 407 and the springs 409, serve to compensate for play and also to compensate for the changed distances of the flanks 114 from one another which result from the variable pitch.

In the above example of the kinematic system 101, the spindle nut 403 is mechanically connected to the movable part 116 of the second linear drive 107 and ensures the controlled movement of the movable part 116 in relation to the spindle 113, wherein the movable part 116 is guided in the support tube 115 to decouple the force absorption, so that the spindle 113, together with the spindle nut 403, has to absorb only axial forces.

1. Medical kinematic system having a virtual fulcrum for a medical robot for guiding a surgical instrument arranged on an instrument holder in a working zone, with a main body, a first linear drive, and a second linear drive for movably arranging an instantaneous fulcrum of the instrument holder in the working zone and simultaneously pivoting the instrument holder about the instantaneous fulcrum, wherein the first linear drive has a first fixed point, for receiving a first linear drive part of the first linear drive on the main body, and a first guide point for guiding a second linear drive part of the first linear drive relative to the main body in a first guide track, and the second linear drive has a second fixed point, for receiving a first linear drive part of the second linear drive, and a second guide point for guiding the second linear drive part of the second linear drive relative to the main body and/or relative to the second linear drive part of the first linear drive in a second guide track, wherein one of the guide tracks has a guide cam, and at least the linear drive guided in the other guide track relative to the guide track with the guide cam has a variable transmission ratio, so that, by means of a transmission arrangement acting mechanically between the first linear drive and the second linear drive and by means of an interaction of a geometry of the guide cam with the at least one variable transmission ratio, the instantaneous fulcrum can be moved in a circular path about the virtual fulcrum, and the instrument holder can be pivoted about the virtual fulcrum.

2. Medical kinematic system according to claim 1, characterized in that the first guide track assigned to the second linear drive part of the first linear drive has the guide cam, wherein in particular the second guide track assigned to the second linear drive part of the second linear drive is substantially rectilinear.

3. Medical kinematic system according to claim 1, characterized in that the second guide track assigned to the second linear drive part of the second linear drive has the guide cam, wherein in particular the first guide track assigned to the second linear drive part of the first linear drive is substantially rectilinear.

4. Medical kinematic system according to claim 3, characterized in that the instrument holder is received on the second linear drive part of the first linear drive so as to be movable substantially in one direction through the virtual fulcrum relative to the second linear drive part of the first linear drive.

5. Medical kinematic system according to claim 1, characterized in that the geometry of the guide cam is arcuate, parabolic, and/or elliptical, wherein a center of curvature of the geometry and/or of a relevant portion of the geometry is arranged starting from the geometry in the direction of the virtual fulcrum.

6. Medical kinematic system according to claim 1, characterized in that the guide cam has at least one sliding link, wherein the guide point has a pin guided in the at least one sliding link.

7. Medical kinematic system according to claim 1, characterized in that the second linear drive is arranged to act substantially parallel to the first linear drive, and/or a first linear drive part of the second linear drive is assigned to the second linear drive part of the first linear drive, wherein in particular the second linear drive part of the second linear drive is driven via the transmission arrangement by means of the first linear drive.

8. Medical kinematic system according to claim 7, characterized in that a lever is assigned to the second linear drive part of one of the linear drives, wherein the lever is connected to the instrument holder in a rotatable, mechanically acting manner for the indirect pivoting of the instrument holder about the instantaneous fulcrum by means of the corresponding linear drive.

9. Medical kinematic system according to claim 1, characterized in that the at least one variable transmission ratio is realized by means of a drive ratio of the first linear drive and/or the second linear drive that can be changed over a length of movement.

10. Medical kinematic system according to claim 9, characterized in that one of the linear drive parts of the first linear drive and/or the second linear drive has a spindle with pitch flanks arranged in a spiral shape along the length of movement and a pitch of the pitch flanks that is variable over the length of movement, wherein the corresponding other linear drive part of the linear drive is assigned a spindle nut guided on the spindle with engagement elements engaging in the pitch flanks.

11. Medical kinematic system according to claim 10, characterized in that the spindle nut has elastic compensating means acting between the engagement elements for compensating for distance differences and/or tolerances occurring along the length of movement between and/or on respective pitch flanks.

12. Medical kinematic system according to claim 1, characterized in that the instrument holder is assigned a third linear drive with a movement axis, running through the virtual fulcrum, for linearly moving the surgical instrument through the virtual fulcrum.

13. Medical kinematic system according to claim 1, characterized in that the main body is assigned a rotary holder with a rotary axis for rotatably connecting the medical kinematic system to a medical robot, wherein the rotary axis is arranged skewed to the movement axis of the first linear drive.

14. A medical robot with a medical kinematic system according to claim 1.

15. A method for operating a medical kinematic system according to claim 1 having a virtual fulcrum, having the following steps:
arranging the medical kinematic system in the working zone so that the medical kinematic system is arranged in the working zone,
guiding the medical kinematic system and/or the medical robot about the virtual fulcrum by means of an interaction of the geometry of the guide cam with the at least one variable transmission ratio, so that the instrument holder is pivoted about the virtual fulcrum,
so that the medical kinematic system is operated with a virtual fulcrum.
